(19) United States
(12) Patent Application Publication
(10) Pub. No.: US 2008/0248124 A1
(43) Pub. Date: Oct. 9, 2008

(54) Process for producing pharmaceutical composition

(75) Inventors: Toru Eguchi, Osaka (JP); Kenji Hasegawa, Osaka (JP)

Correspondence Address: FOLEY AND LARDNER LLP;SUITE 500, 3000 K STREET NW, WASHINGTON, DC, 20007, US

(73) Assignee: Sunstar Kabushiki Kaisha

(21) Appl. No.: 11/730,746

(22) Filed: Apr. 3, 2007

(51) Int. Cl. A61K 9/52; A61K 31/65; A61P 1/02; A61K 47/06

(52) U.S. Cl. 424/494; 514/152; 514/772.4; 514/777

(57) ABSTRACT

The present invention relates to a process for producing a pharmaceutical composition which can stably contain an active ingredient unstable against water and can sustained-release such the active ingredient for a long period of time by remaining at an administrated portion as well as a pharmaceutical composition produced by the same. Specifically, the present invention relates to a process for producing a pharmaceutical composition, comprising steps of: mixing and heating a first phase, prepared by mixing a polyhydric alcohol and a salt, and a second phase containing a water-soluble polymer under a reduced pressure, before evaporating substantially all water in the first phase by mixing and heating a mixture of first and second phases under a reduced pressure or after evaporating substantially all water in the first phase by mixing and heating the first phase under a reduced pressure; and adding a third phase containing an active ingredient unstable against water and mixing them to obtain the pharmaceutical composition, as well as a pharmaceutical composition produced the same.

### FIELD OF THE INVENTION

The present invention relates to a process for producing a pharmaceutical composition. More specifically, the present invention relates to a process for producing a pharmaceutical composition which can stably contain an active ingredient unstable against water and can remain at an administered portion, thereby, sustainedly release such the active ingredient for a long period of time as well as a pharmaceutical composition produced by the same.

### BACKGROUND OF THE INVENTION

Among active ingredients of the pharmaceutical composition such as antibiotics and anti-inflammatories, there are some active ingredients, which have a high efficacy, but are unstable and, therefore, are formulated into pharmaceutical compositions with limitation. For example, tetracycline and macrolide antibiotics are active ingredients having a broad antibacterial spectrum, but they are substances affected by water, heat or an additive and are easily denatured, when they are formulated into compositions. Therefore, various techniques for stably formulating these antibiotics into the pharmaceutical compositions have been previously studied.

For example, JP 52-90616 A discloses an aqueous solution for injection, aiming at stabilizing the tetracycline antibiotics such as oxytetracycline, doxycycline, tetracycline, chlortetracycline or salts thereof by chelating it with an alkaline-earth metal compound such as a magnesium compound in an aqueous solution of 2-pyrrolidone. Moreover, JP 53-94028 A discloses a pharmaceutical composition, aiming at stabilizing oxytetracycline by incorporating into the composition an alkaline-earth metal ion, polyvinylpyrrolidone and aliphatic amide and adjusting a pH of the composition to 5.0-7.5. Furthermore, U.S. Pat. No. 3,335,055 discloses a method for stabilizing tetracycline with a magnesium ion and a pyridine derivative such as isonicotinic acid amide, etc.

Moreover, JP H02-34325 B and JP H07-29930 B disclose a pharmaceutical composition which can stably contain one of tetracycline antibiotics, minocycline, and can exert the continuous effect of minocycline for a long period of time by formulating minocycline with a magnesium compound, a water-soluble polymer, a polyhydric alcohol, a methacrylate copolymer and a solubilizing agent.

However, there has been a problem that production of the pharmaceutical composition exerting such effects is difficult. For example, when such the pharmaceutical composition is simply produced according to a conventional procedure, water is mixed into the pharmaceutical composition. Water mixed into the composition cannot be sufficiently removed even by simply heating the composition at a high temperature for a long period of time because the water binds to a highly hydratable ingredient in the composition. In addition, there has been a problem that a molecular chain of the polymer in the composition is sometimes cut, and that the ingredient in the composition is denatured due to a chemical reaction between the ingredients, etc.

On the other hand, when the pharmaceutical composition which cannot remain at an administered portion is administered to a periodontal disease portion, for example, a periodontal pocket, a concentration of the active ingredient in the periodontal pocket cannot be maintained for a long period of time due to a flow of saliva, foods, drinks, etc. in an oral cavity, even though a high concentration of the active ingredient temporarily remains at the periodontal pocket and a certain extent of treatment effects can be obtained. Accordingly, in order to obtain the treatment effect, the active ingredient must be administered repeatedly within a short period and it burdened a patient.

An object of the present invention is to provide a process for producing a pharmaceutical composition which can stably incorporate an active ingredient unstable against water and can remain at an administered portion, thereby, sustainedly release it for a long period of time.

The present inventors have intensively studied in order to solve the problems, and found that the problems can be solved by mixing a water-soluble polymer with a polyhydric alcohol base containing a salt before or after mixing and heating the base under a reduced pressure, and then adding an active ingredient after cooling the mixture, and further by combining the mixture with a film-forming agent dissolved in an organic solvent, which resulted in completion of the present invention.

That is, the present invention relates to:

[1] A process for producing a pharmaceutical composition, comprising steps of:

mixing and heating a first phase, prepared by mixing following ingredients (A) and (B), and a second phase containing an ingredient (C) under a reduced pressure, before evaporating substantially all water in the first phase by mixing and heating a mixture of first and second phases under a reduced pressure or after evaporating substantially all water in the first phase by mixing and heating the first phase under a reduced pressure; and

adding a third phase containing an ingredient (D) to the mixture to obtain the pharmaceutical composition,

(A) one or more of polyhydric alcohols selected from the group consisting of glycerin, ethylene glycol, diethylene glycol, propylene glycol, dipropylene glycol, hexylene glycol, 1,5-pentanediol, 1,3-butylene glycol, and polyethylene glycol;

(B) one or more of salts selected from the group consisting of magnesium, calcium, and barium salts and hydrous salts thereof;

(C) one or more of water-soluble polymers selected from the group consisting of hydroxyethyl cellulose, hydroxymethyl cellulose, methyl cellulose, hydroxypropyl cellulose, hydroxypropylmethyl cellulose, carboxymethylethyl cellulose, sodium carboxymethy cellulose, sodium alginate, polyvinyl alcohol, polyvinylpyrrolidone, carrageenan, xanthan gum, locust bean gum, guar gum, tragacanth gum, starch and succinoglucan; and

(D) one or more of active ingredients unstable against water;

[2] The process according to [1], wherein the first and second phases are mixed and heated after evaporating substantially all water in the first phase by mixing and heating the first phase under a reduced pressure;
[3] The process according to [1], wherein the salt (B) is one or more selected from the group consisting of magnesium chloride, magnesium acetate, magnesium sulfate, magnesium nitrate, magnesium carbonate, magnesium gluconate, magnesium oxide, magnesium hydroxide and hydrous salts thereof;
[4] The process according to [1], wherein the water-soluble polymer (C) is one or more of cellulose derivatives selected from the group consisting of hydroxyethyl cellulose, hydroxymethyl cellulose, methyl cellulose, hydroxypropyl cellulose, hydroxypropylmethyl cellulose, carboxymethylethyl cellulose and sodium carboxymethy cellulose;
[5] The process according to [1], wherein the active ingredient (D) is one or more of tetracycline antibiotics selected from the group consisting of tetracycline, minocycline, doxycycline, oxytetracycline, chlortetracycline and pharmaceutically acceptable salts thereof;
[6] The process according to [1], further comprising a step of adding and mixing a fourth phase, prepared by mixing following ingredients (E) and (F):

(E) methacrylate copolymer; and

(F) one or more of organic solvents selected from the group consisting of triacetin, tributyrin, ethylene glycol diacetate, diethyl sebacate, diethyl phthalate, dibutyl phthalate, diisopropyl adipate, dibutyl succinate, triethyl citrate, N-methyl-2-pyrrolidone and propylene carbonate;

[7] The process according to [6], comprising steps of:

(1) mixing and heating the first phase under a reduced pressure to evaporate substantially all water therein;

(2) adding the second phase to the first phase and mixing and heating them;

(3) adding the third phase to the mixture and mixing them; and

(4) adding the fourth phase to the mixture and mixing them to obtain a pharmaceutical composition;

[8] The process according to [6], comprising steps of:

(1) mixing and heating the first phase under a reduced pressure to evaporate substantially all water therein;

(2) adding the second phase to the first phase and mixing and heating them;

(3) adding the fourth phase to the mixture and mixing them; and

(4) adding the third phase to the mixture and mixing them to obtain a pharmaceutical composition;

[9] The process according to [6], comprising steps of:

(1) mixing and heating the first and second phases under a reduced pressure to evaporate substantially all water therein;

(2) adding the third phase to the mixture and mixing them; and

(3) adding the fourth phase to the mixture and mixing them to obtain a pharmaceutical composition;

[10] The process according to [6], comprising steps of:

(1) mixing and heating the first and second phases under a reduced pressure to evaporate substantially all water therein;

(2) adding the fourth phase to the mixture and mixing them; and

(3) adding the third phase to the mixture and mixing them to obtain a pharmaceutical composition;

[11] The process according to [6], wherein the polyhydric alcohol (A) and the organic solvent (F) are immiscible;
[12] A pharmaceutical composition produced by a process as defined in [1];
[13] A pharmaceutical composition produced by a process as defined in [6], which produces a microcapsule complex by coming in contact with water;
[14] The pharmaceutical composition according to [12], which is a dental pharmaceutical composition; and
[15] The pharmaceutical composition according to [14], which is a pharmaceutical composition for treating periodontal diseases.

According to the invention of [1], there can be provided a pharmaceutical composition which can stably formulate an active ingredient that is unstable against water.

According to the invention of [2], as compared with the invention of [1], the active ingredient that is unstable against water can be more stably formulated in the pharmaceutical composition because water contained in the ingredients (A) and (B) can be removed more surely.

According to the invention of [3], as compared with the invention of [1] or [2], the active ingredient can be more stably formulated, because the active ingredient unstable against water is more stabilized by a base containing the magnesium compound.

According to the invention of [4], as compared with any one invention of [1] to [3], a more suitable hydro gel and a more stabilized microcapsule complex can be obtained. Moreover, when the cellulose derivative is used, there can be easily prepared the pharmaceutical composition which is adapted to an administration portion and an administration object, because a particle diameter of the hydro gel in the produced microcapsule complex may be controlled by varying an amount of the cellulose derivative.

According to the invention of [5], as compared with any one invention of [1] to [4], there can be provided a totally higher medical contribution in comparison with a case of formulations of other antibiotics that are unstable against water, because the invention is directed to formulation of antibiotics which are widely used.

According to the invention of [6], as compared with any one invention of [1] to [5], side effects due to release of a large amount of the active ingredient can be suppressed and the effect of the active ingredient can be continuously exerted for a longer period of time, because the stably formulated active ingredient can be sustainedly released from the composition for a longer period of time.

According to the inventions of [7] to [10], as compared with the invention of [6], a production step properly corresponding to the ingredient to be used can be applied and, thereby, a flexibility of a production process can be broadened, because the same effect can be obtained irrespective of an order of addition of the ingredients.

According to the invention of [11], as compared with any one invention of [6] to [10], sustained-releasing of the active ingredient for a long period of time is more assured, because a microcapsule complex in which a hydro gel formed from the polyhydric alcohol and the water-soluble polymer is coated with a methacrylate copolymer film is more stabilized.

According to the invention of [12], there can be provided a pharmaceutical composition having aforementioned advantages of the inventions of [1] to [11];

According to the invention of [13], as compared with any one invention of [6] to [11], the active ingredient can be released more stably and sustainedly for a long period of time, because the active ingredient retained in a non-aqueous inside of the microcapsule complex can be gradually released to an outside of the microcapsule complex.

According to the invention of [14], as compared with the invention of [12] or [13], there can be provided a pharmaceutical composition exerting an excellent effect, particularly, for a dental use.

According to the invention of [15], as compared with the invention of [14], there can be provided a pharmaceutical composition exerting an excellent effect, particularly, for a therapeutic use of periodontal diseases.

### BRIEF DESCRIPTION OF THE DRAWINGS

FIG. 1 is a schematic diagram illustrating a vessel used in Experimental Example 4.

FIG. 2 is a graph showing a result of an elution experiment (Experimental Example 4).

FIG. 3 is a graph showing a result of measurement of a particle diameter of a pharmaceutical composition (Experimental Example 6).

FIG. 4 is a scanning electron microphotograph showing a state of the pharmaceutical composition contacted with water.

FIG. 5 is a graph showing a relationship between a particle diameter of the pharmaceutical composition and a release rate constant of the active ingredient.

FIG. 6 is a graph showing a change in a concentration of the active ingredient with time after administration of the pharmaceutical composition into a human gingival groove.

### DETAILED DESCRIPTION OF THE PREFERRED EMBODIMENTS

In the first embodiment, the present invention provides a process for producing a pharmaceutical composition.

In the process of the present invention, a first phase of a mixture of a polyhydric alcohol (A) and a salt (B) and a second phase containing a water-soluble polymer (C) are mixed and heated, and then a third phase containing an active ingredient (D) unstable against water is added to the mixture and mixed.
A polyhydric alcohol (A) used in the present invention dissolves the salt and forms a hydro gel containing the active ingredient and the salt with the water-soluble polymer which is added later, and examples include glycerin, ethylene glycol, diethylene glycol, propylene glycol, dipropylene glycol, hexylene glycol, 1,5-pentanediol, 1,3-butylene glycol, polyethylene glycol, etc. Such the polyhydric alcohol may be used alone or in a combination of two or more. Among them, a polyhydric alcohol having a high water content and hydration force such as glycerin, propylene glycol and 1,3-butylene glycol is preferable. Glycerin is particularly preferable because it has a particularly high water content and a low irritation to a human body, in addition to that an effect of the invention is significantly exerted by removing water in glycerin. An amount of the polyhydric alcohol is about 50 to 85% by weight based on a total weight of the pharmaceutical composition. When the amount of the polyhydric alcohol is not within the range, a stable hydro gel cannot be formed and the active ingredient cannot be stably formulated in the composition.

A salt (B) used in the present invention is for stabilizing the active ingredient (D) unstable against water in the pharmaceutical composition, and examples include a magnesium salt such as magnesium chloride, magnesium acetate, magnesium sulfate, magnesium nitrate, magnesium carbonate, magnesium gluconate, magnesium oxide, magnesium hydroxide etc; a calcium salt such as calcium chloride, calcium sulfate, calcium nitrate, calcium gluconate, calcium malate, calcium lactate, calcium oxide, calcium hydroxide etc; a barium salt such as barium chloride, barium sulfate, barium nitrate etc; and hydrous salts thereof. Such the salt may be used alone or in a combination of two or more. Among them, the magnesium salt is preferable because it suitably stabilizes the active ingredient unstable in water, and hydrous salts thereof are also preferable because it is advantageous for forming the hydro gel with other ingredients and bound water is removed. Magnesium chloride or a hexahydrate thereof is particularly preferable because it is conventionally used in the pharmaceutical field and bound water is removed. An amount of the salt is about 0.5-10% by weight based on a total weight of the pharmaceutical composition and is about 0.1-10 fold weight based on a weight of the active ingredient unstable against water. When the amount of the salt is not within the range, the active ingredient cannot be formulated with stability in the pharmaceutical composition.

A water-soluble polymer (C) used in the present invention forms a hydro gel with the polyhydric alcohol and examples include hydroxyethyl cellulose, hydroxymethyl cellulose, methylcellulose, hydroxypropyl cellulose, hydroxypropylmethyl cellulose, carboxymethylethyl cellulose, sodium carboxymethyl cellulose, sodium alginate, polyvinyl alcohol, polyvinylpyrrolidone, carrageenan, xanthan gum, locust bean gum, guar gum, tragacanth gum, starch, succinoglucan, etc. Such the water-soluble polymer may be used alone or in a combination of two or more. Among them, the polymer having a hydroxyl group is preferable because it forms the hydro gel containing the active ingredient, cellulose derivatives are more preferable, and hydroxyethyl cellulose is particularly preferable. An amount of the water-soluble polymer is about 0.1-20% by weight, preferably about 0.5-10% by weight based on a total weight of the pharmaceutical composition and is about 0.2-50 parts by weight, preferably about 1-10 parts by weight based on 100 parts by weight of the polyhydric alcohol. When the amount of the polyhydric alcohol is not within the range, a stabilized hydro gel cannot be formed and the active ingredient cannot be formulated with stability in the pharmaceutical composition.

Moreover, as described below, in an embodiment where the methacrylate copolymer and the organic solvent are additionally added to the composition, a particle diameter of the hydro gel in the microcapsule complex formed when the produced pharmaceutical composition contacts with water may greatly vary depending upon an amount of the water-soluble polymer. When an amount of the water-soluble polymer is great, the particle diameter of the hydro gel in the formed microcapsule complex becomes large.

In this step of the production process, a first phase is prepared by mixing and heating the polyhydric alcohol and the salt, a second phase containing the water-soluble polymer is added to the first phase, and they are mixed and heated. Before mixing the first phase and the second phase, substantially all water contained in the first phase is removed by heating the mixture usually to not lower than about 80° C., preferably to about 90-100° C. under a reduced pressure of not higher than about 100 mmHg, preferably not higher than about 80 mmHg. When a temperature is lower than about 80° C., all of the salts do not dissolve into the polyhydric alcohol. On the other hand, when a pressure is not reduced to not higher than about 100 mmHg, it becomes difficult to remove substantially all water.

Moreover, from a viewpoint of stability of the water-soluble polymer, such removal of water under a reduced pressure is preferably conducted only on the first phase before mixing the first phase and the second phase.

The phrase “removing substantially all water” used herein means that an amount of water contained in the mixture becomes not greater than about 3% by weight, preferably not greater than about 2% by weight, and more preferably not greater than about 1% by weight. Preferably, an to amount of water contained in the mixture may be calculated by measuring an amount of trapped water which has been removed by suction, and subtracting the measured amount from an amount of water contained in the mixture before suction. Thereby, the amount of water contained in the mixture may be exactly calculated, and a concentration of other ingredient, particularly the active ingredient, may be exactly and easily adjusted by adding other ingredients in the same amount as that of trapped water.

The water-soluble polymer of the second phase becomes difficult in some cases to be mixed uniformly with the first phase at a high temperature when it is solely added thereto, depending upon a kind of the polyhydric alcohol of the first phase. Therefore, in such the case, the water-soluble polymer may be added to the first phase after dispersing it in the polyhydric alcohol at ambient temperature. This polyhydric alcohol may be the same or different from that used in the first phase.

Then, the first phase and the second phase are mixed and heated to about 95-180° C., preferably about 100-140° C. When a temperature of the mixture is lower than about 95° C., it becomes difficult to dissolve the water-soluble polymer in a shorter period. On the other hand, when a temperature of the mixture is higher than about 180° C., degradation of the water-soluble polymer may be caused and, thereby, the active ingredient cannot be stably formulated in the pharmaceutical composition. Moreover, this step can be conducted under an atmospheric or reduced pressure. When the mixing is conduced under an atmospheric pressure, denaturation of the ingredient such as degradation of the water-soluble polymer can be suppressed because the condition becomes milder than that under a reduced pressure. On the other hand, when the mixing is conduced under a reduced pressure, water can be further removed during this mixing. Preferably, the mixing is conducted under an atmospheric pressure, followed by under a reduced pressure. Thereby, not only the water-soluble polymer can be mixed mildly under an atmospheric pressure, but also bubbles in the mixture can be removed simultaneously with removal of water under a reduced pressure and, thereby, stability of the pharmaceutical composition can be more enhanced. Moreover, the mixture is preferably subjected to a reduced pressure after dissolution of the water-soluble polymer is confirmed. An extent of a reduced pressure may be properly set, and it is preferably not higher than about 100 mmHg, more preferably not higher than about 80 mmHg. Moreover, reduction of a pressure may be initiated at an initial temperature not lower than about 65° C., preferably not lower than about 80° C. and, thereafter, a temperature may be lowered to not higher than about 75° C., preferably not higher than 65° C. while a pressure is reduced.

Next, after it is confirmed that the water-soluble polymer has been uniformly dissolved, a temperature of the mixture is lowered to not higher than about 75° C., preferably not higher than about 70° C., and more preferably not higher than about 65° C., and the active ingredient or the mixture of the active ingredient and the polyhydric alcohol are mixed. Thereafter, mixing and degassing under a reduced pressure affords a pharmaceutical composition consisting of the hydro gel. When the active ingredient is added at a temperature higher than about 75° C., degradation of the active ingredient is caused and a potency of the pharmaceutical composition is decreased. Moreover, when the mixture is not degassed, stability of the pharmaceutical composition is decreased.

An active ingredient (D) unstable against water used in the present invention is an active ingredient which becomes unstable in the presence of water. The phrase “an active ingredient unstable against water” as used herein means such a compound that, when it is dissolved in or mixed with purified water at room temperature and stored at 25° C. for seven days, a ratio of an un-denatured ingredient is lowered to not greater than 95%, preferably to not greater than 90%. Examples of the active ingredient unstable against water include an anti-bacterial agent such as tetracycline, penicillin, carbapenem, cephem, monobactum, aminoglycoside and macrolide antibiotics; an anti-fungal agent such as polyene, azole, echinocandin and pyrimidine antibiotics; and an anti-inflammatory such as steroids and non-steroids, etc. Among them, one or more tetracycline antibiotics, which are remarkably stabilized by a salt of bivalent metal ion, such as those selected from the group consisting of minocycline, doxycycline, tetracycline, oxytetracycline, chlortetracycline and pharmaceutically acceptable salts thereof are preferable, and minocycline is most preferable.

Such the active ingredients may be used alone or in a combination of two or more. An amount of the active ingredient may vary depending upon a desired effect, and is generally about 0.1-10.0% by weight based on a total weight of the composition.

Moreover, in the case where the active ingredient has a powder form or the like at ambient temperature, when it is solely added to the mixture of the first phase and the second phase, it becomes difficult to uniformly mix them in some cases. Therefore, in such the case, the active ingredient may be added to the mixture of the first phase and the second phase after it is dissolved or dispersed in the polyhydric alcohol at ambient temperature. The polyhydric alcohol used in this step may be the same or different from that used in the previous step. Moreover, an amount of the polyhydric alcohol may be properly set so long as the active ingredient may be dispersed therein, but preferably it is an amount replenishing an amount of water removed. That is, in order to adjust a concentration of the active ingredient in the pharmaceutical composition, an amount of removed water may be measured and the same amount of polyhydric alcohol as that of removed water may be used for dissolving or dispersing the active ingredient.

Furthermore, in the process of the present invention, in addition to the aforementioned ingredients essential for forming the hydro gel, a fourth phase in which the methacrylate copolymer (E) and the organic solvent (F) are mixed may be added to impart a sustained-releasing property to the active ingredient stably stored in the hydro gel.

A methacrylate copolymer (E) used in the present invention is a film-forming agent for forming a film on the hydro gel which is formed from the salt, the active ingredient, the polyhydric alcohol and the water-soluble polymer. Examples of the methacrylate copolymer include aminoalkyl methacrylate copolymer (a copolymer of methyl methacrylate with butyl methacrylate and dimethylaminoethyl methacrylate (for example, Eudragit®E, Pharma Polymers)), aminoalkyl methacrylate copolymer (a copolymer of ethyl acrylate with methyl methacrylate and methacrylate ethyl trimethylammonium chloride (for example, Eudragit®RS, Pharma Polymers)), etc. Such the methacrylate copolymers may be used alone or in a combination of two or more. An amount of the methacrylate copolymer is about 0.5-10% by weight based on a total weight of the pharmaceutical composition. When the amount is less than 0.5% by weight, sustained-releasing of the active ingredient becomes difficult. On the other hand, when the amount is greater than 10% by weight, a viscosity of the composition becomes high and production of the pharmaceutical composition becomes difficult.

An organic solvent (F) used in the present invention is an organic solvent which can dissolve the methacrylate copolymer but is immiscible with the polyhydric alcohol used in the foregoing step. By the organic solvent, the methacrylate copolymer may be present in the dissolved state at an outside of the hydro gel formed from the first to third phases. Upon contact of the produced pharmaceutical composition with water, the methacrylate copolymer which has been dissolved becomes an insoluble porous film covering the hydro gel, to form a microcapsule complex. Examples of the organic solvent include an ester of a lower polyhydric alcohol with a lower fatty acid, such as triacetin, tributyrin, ethylene glycol diacetate etc., an ester of a lower alcohol with a dicarboxylic acid, such as diethyl sebacate, diethyl phthalate, dibutyl phthalate, diisopropyl adipate, dibutyl succinate etc., or the like. Such the organic solvents may be used alone or in a combination of two or more. An amount of the organic solvent is preferably about 5-25% by weight based on a total weight of the pharmaceutical composition. When the amount of the organic solvent is not within the range, sustained-releasing of the active ingredient becomes difficult because a suitable microcapsule complex is not formed upon contact of the pharmaceutical composition with water. Now, the microcapsule complex used herein refers to a capsule in which a plurality of micron-sized hydro gels are complexed with the film.

A blending ratio of the fourth phase containing the methacrylate copolymer (E) and the organic solvent (F) is preferably about 1-100 parts by weight, more preferably about 5-50 parts by weight, and most preferably about 10-20 parts by weight relative to 100 parts by weight of the hydro gel formed from the first to third phases. Within this blending ratio, a particle diameter of the hydro gel in the microcapsule complex formed by contact with water may be controlled. When the blending ratio of the organic solvent relative to the methacrylate copolymer becomes great, the particle diameter of the hydro gel in the microcapsule complex becomes large. Moreover, a release rate constant of the active ingredient from the microcapsule complex becomes great with increase in the particle diameter. When the particle diameter of the hydro gel in the microcapsule complex is increased, a viscosity of the pharmaceutical composition is reduced and retention of the pharmaceutical composition at an administered portion is reduced. When the blending ratio is not the range, stability of the microcapsule complex is deteriorated and sustained-releasing of the active ingredient becomes difficult.

Furthermore, in the case where an amount range of the aforementioned ingredients is defined herein, when the total amount of respective ingredients exceeds 100% by weight, of course, it should be understood that it means that respective ingredients are added in an amount within the defined range such that the total amount of the ingredients becomes not greater than 100% by weight.

In the second embodiment, the present invention provides a pharmaceutical composition produced by the aforementioned production process.

The pharmaceutical composition of the present invention is substantially free from water. An amount of water contained in the pharmaceutical composition is preferably not greater than about 2% by weight, and more preferably not greater than about 1% by weight. When the amount of water is greater than about 2% by weight, a potency of the active ingredient cannot be maintained for a long period of time.

The pharmaceutical composition produced by the process of the present invention usually has a paste form or an ointment, but it may be produced in another form such as liquid-, gel-, cream-, film-, chip-, particle-, cube-, sphere- or sheet-like form, etc. so that the potency of the active ingredient contained therein is properly exerted. A film- or sheet-like pharmaceutical composition may be formed by using a film producing apparatus, or by a wet process by casting a paste-like pharmaceutical composition obtained by the process of the present invention on a plane, and air-drying and molding it, according to the conventional procedure. The pharmaceutical composition of these forms containing the methacrylate copolymer and the organic solvent forms the microcapsule complex upon contact with water, and sustained-releasing of the active ingredient therefrom is achieved.

And, the pharmaceutical composition of the present invention may be administered via any route depending upon a medical effect of the active ingredient contained and an object of administration. The administration route includes, for example, topical, intravenous, subcutaneous, intramuscular, intra-orbital, ocular, intraventricular, endocranial, intra-capsular ligament, intraspinal, intracisternal, intra abdomina, intranasal, oral, buccal, rectal and intravaginal routes.

For example, in the case where minocycline or a pharmaceutically acceptable salt thereof is used as the active ingredient unstable against water, it may be produced into a dental pharmaceutical composition, and the dental pharmaceutical composition may be produced as a pasty ointment, a film or sheet shape or a chip shape pharmaceutical composition which is administered topically. In the case where the dental pharmaceutical composition has a pasty ointment form, for example, it may be conveniently administered to an affected portion such as a periodontal pocket with a syringe for use in application for periodontal diseases. In addition, in the case where the composition has a film or sheet form or a chip shape, it may be inserted into a narrow periodontal pocket.

Therefore, in the case where the dental pharmaceutical composition is formulated into such forms, occurrence of a systemic side effect which has been previously observed upon oral administration, for example, a digestive system side effect such as anorexia, nausea and diarrhea, a biochemical side effect such as thrombocytopenia and eosinophilia, or superinfection can be suppressed and a medicinal effect can be efficiently exerted. Moreover, by sustained-releasing of minocycline for a long period of time, re-administration of the pharmaceutical composition to a patient within a short period becomes un-necessary and a burden on the patient is reduced.

Furthermore, in addition to ingredients described above, ingredients which are conventionally contained in the pharmaceutical composition such as coloring agents, flavoring agents, surface active agents, excipients, etc. may be contained in the pharmaceutical composition of the present invention, so long as they does not deteriorate the effects of the invention.

Next, the invention will be illustrated in more detail referring to working examples, but it is intended to illustrative, and should not to be construed to limit the scope of the invention thereto.

### PRODUCTION EXAMPLE 1 (MICROCAPSULE COMPLEX-FORMING FORMULATION)

11.2 kg of glycerin was placed in a 20 L planetary mixer equipped with a vacuum pump, and 1 kg of magnesium chloride hexahydrate was dispersed therein. The mixture was heated to 90-100° C. at not higher than 100 mmHg to dissolve and mix it to remove substantially all water contained in glycerin and magnesium chloride hexahydrate. Removed water was captured with a trap, and an amount thereof was measured. After the mixture was returned to an atmospheric pressure, another mixture in which 0.8 kg of hydroxy ethylcellulose had been dispersed in 2.8 kg of glycerin was added to the mixture, which was heated to not lower than 130° C. and stirred. After uniform dissolution of hydroxy ethylcellulose was confirmed by viewing, the mixture was cooled while it was degassed under a reduced pressure at not higher than 100 mmHg. After a temperature of the mixture became not higher than 65° C., a mixture in which 0.4 kg of minocycline hydrochloride had been dispersed in glycerin (the sum of weight equal to that of water trapped and 1 kg (the sum of weight was adjusted so that the total amount became 20 kg)) was added, followed by stirring. After uniform dissolution of minocycline hydrochloride was confirmed by viewing, a solution in which 0.4 kg of Eudragit®RS had been dissolved in 2.4 kg of triacetin was added, followed by further stirring. Thereafter, the mixture was stirred uniformly and degassed to obtain a paste-like pharmaceutical composition containing minocycline hydrochloride as an active ingredient (Composition 1).

### PRODUCTION EXAMPLE 2 (MICROCAPSULE COMPLEX-FORMING FORMULATION)

10.5 kg of glycerin was placed in a 20 L planetary mixer equipped with a vacuum pump, and 1 kg of magnesium chloride hexahydrate and 0.8 kg of hydroxyethyl cellulose were dispersed therein. A mixture was heated to about 130° C. at not higher than 100 mmHg to dissolve and mix it to remove substantially all water contained in glycerin and magnesium chloride hexahydrate, thereby, a hydro gel was prepared. Removed water was captured with a trap, and an amount thereof was measured. After the mixture was returned to an atmospheric pressure, the mixture was cooled. After a temperature of the mixture became not higher than 65° C., a mixture in which 0.6 kg of tetracycline hydrochloride had been dispersed in glycerin (the sum of weight equal to that of water trapped and 1.5 kg (the sum of weight was adjusted so that the total amount became 20 kg)) was added, followed by stirring. After uniform dissolution of minocycline hydrochloride was confirmed by viewing, a solution in which 0.4 kg of Eudragit®RS had been dissolved in 2.4 kg of triacetin was added, followed by further stirring. Thereafter, the mixture was stirred uniformly and degassed to obtain a paste-like pharmaceutical composition containing tetracycline hydrochloride as an active ingredient (Composition 2).

### PRODUCTION EXAMPLE 3 (HYDRO GEL FORMULATION)

13.2 kg of glycerin was placed in a 20 L planetary mixer equipped with a vacuum pump, and 1.2 kg of magnesium chloride hexahydrate was dispersed therein. A mixture was heated to 90-100° C. at not higher than 100 mmHg to dissolve and mix it to remove substantially all water contained in glycerin and magnesium chloride hexahydrate. Removed water was captured with a trap, and an amount thereof was measured. After the mixture was returned to an atmospheric pressure, a mixture in which 1 kg of hydroxyethyl cellulose had been dispersed in 3.2 kg of glycerin was added, and the mixture was heated to not higher than 130° C., followed by stirring. After uniform dissolution of hydroxy ethylcellulose was confirmed by viewing, the mixture was cooled while it was degassed under a reduced pressure of not higher than 100 mmHg. After a temperature of the mixture became not higher than 65° C., a mixture in which 0.4 kg of doxycycline hydrochloride had been dispersed in glycerin (the sum of weight equal to that of water trapped and 1 kg (the sum of weight was adjusted so that the total amount became 20 kg)) was added, followed by stirring. Thereafter, the mixture was mixed uniformly and degassed to obtain a paste-like pharmaceutical composition containing doxycycline hydrochloride as an active ingredient (Composition 3).

### PRODUCTION EXAMPLE 4

According to the process of Production Example 1 except for using the same amount of ethylene glycol in place of glycerin, a pharmaceutical composition was produced.

### PRODUCTION EXAMPLE 5

According to the process of Production Example 1 except for using the same amount of magnesium sulfate heptahydrate in place of magnesium chloride hexahydrate, a pharmaceutical composition was produced.

### PRODUCTION EXAMPLE 6

According to the process of Production Example 1 except for using the same amount of xanthan gum in place of hydroxyethyl cellulose, a pharmaceutical composition was produced.

### PRODUCTION EXAMPLE 7

According to the process of Production Example 1 except for using the same amount of dibutyl phthalate in place of triacetin, a pharmaceutical composition was produced.

### PRODUCTION EXAMPLE 8

According to the process of Production Example 1 except for using the same amount of triethyl citrate in place of triacetin, a pharmaceutical composition was produced.

### COMPARATIVE PRODUCTION EXAMPLE 1

A pharmaceutical composition from which water had not been removed sufficiently was obtained by rendering insufficient a degree of a reduced pressure and a temperature upon dissolution and mixing of glycerin and magnesium chloride hexahydrate in Production Example 1 (Comparative Composition 1).

### COMPARATIVE PRODUCTION EXAMPLE 2

A pharmaceutical composition from which water had not been removed sufficiently was obtained by rendering insufficient a degree of a reduced pressure and a temperature upon dissolution and mixing of glycerin and magnesium chloride hexahydrate in Production Example 3 (Comparative Composition 2).

### EXPERIMENTAL EXAMPLE 1

### Measurement of Amount of Water in Pharmaceutical Composition

Each of the Composition 1 and the Comparative Composition 1 was experimentally produced three times, and an amount of water contained in each composition was measured with a Karl-Fischer moisture titrator. The result thereof is shown in Table 1.

TABLE 1

| | | Comparative |
|---|---|---|
| | Composition 1 | Composition 1 |
| Experimentally produced product 1 | 0.56% | 5.03% |
| Experimentally produced product 2 | 0.62% | 6.25% |
| Experimentally produced product 3 | 0.65% | 6.68% |

As apparent from Table 1, it was confirmed that in Composition 1 produced according to the process of the present invention, an amount of water was low and water had been sufficiently removed in any of experimentally produced products. On the other hand, it was found that in Comparative Composition 1 in which removal of water was insufficient, a larger amount of water was contained as compared with the Composition 1.

### EXPERIMENTAL EXAMPLE 2

### Stability Experiment of Active Ingredient

Each of about 0.5 g of Compositions 1 and 3 and Comparative Compositions 1 and 2 was filled into a dental syringe of about 0.5 mL volume (outer cylinder: made from polypropylene, piston body made from polypropylene, piston tip rubber made from silicone rubber), and a ratio of potency remained of the active ingredient in the composition was measured after storage for a predetermined period at 15 or 30° C. The remaining activity rate was calculated based on an amount of the active ingredient measured with HPLC (pump: Model 510 (Nihon Millipore K.K.), column: Vydac C-18 (Grace Vydac), mobile phase: 16% aqueous isopropyl alcohol solution containing 50 mL of diethanolamine and 1 mM EDTA, adjusted to pH 8 with 1N aqueous sodium hydroxide solution, detector: M490 (Nihon Millipore K.K.), detection wavelength: 345 nm, flow rate: 1.0 ml/sec, injection amount: 50 μl). The result thereof is shown in Table 2.

TABLE 2

| | Compo- | | Comparative | Comparative |
|---|---|---|---|---|
| | sition 1 | Composition 3 | Composition 1 | Composition 2 |
| 15° C., | 100% | 100% | 99% | 99% |
| 1 month | | | | |
| 15° C., 3 | 99% | 99% | 96% | 97% |
| months | | | | |
| 15° C., 6 | 98% | 99% | 89% | 90% |
| months | | | | |
| 30° C., | 99% | 99% | 96% | 95% |
| 1 month | | | | |
| 30° C., 3 | 97% | 98% | 86% | 88% |
| months | | | | |
| 30° C., 6 | 95% | 96% | 71% | 69% |
| months | | | | |

As apparent from Table 2, it was found that not less than 95% of an activity of the active ingredients remained even after storage for 6 months at 30° C., in Compositions 1 and 3 (minocycline hydrochloride and doxycycline hydrochloride, respectively) produced according to the process of the present invention. On the other hand, it was found that the activity in the Comparative Compounds 1 and 2 was lowered to about 70% after storage for 6 months at 30° C.

### EXPERIMENTAL EXAMPLE 3

### Stability Test of Active Ingredient

A stability test of the active ingredient was performed according to the aforementioned method of Experimental Example 2 except that each of the composition and comparative composition was filled into an aluminum tube in place of the dental syringe. The result thereof is shown in Table 3.

TABLE 3

| | Compo- | Compo- | Comparative | Comparative |
|---|---|---|---|---|
| | sition 1 | sition 3 | composition 1 | composition 2 |
| 15° C., 1 month | 100% | 100% | 98% | 98% |
| 15° C., 3 months | 100% | 99% | 94% | 95% |
| 15° C., 6 months | 98% | 98% | 88% | 86% |
| 30° C., 1 months | 99% | 99% | 90% | 91% |
| 30° C., 3 months | 97% | 97% | 84% | 82% |
| 30° C., 6 months | 96% | 95% | 68% | 65% |

As apparent from Table 3, it was found that not less than 95% of an activity of the active ingredients remained even after storage for 6 months at 30° C., in Compositions 1 and 3 produced according to the process of the present invention. On the other hand, it was found that the activity in Comparative Compounds 1 and 2 was lowered to less than 70% after storage for 6 months at 30° C.

Moreover, from comparison with the result of Experimental Example 2, it was demonstrated that the stability of the active ingredient in the composition produced according to the process of the present invention is not influenced by a vessel material.

### EXPERIMENTAL EXAMPLE 4

### Elution Test

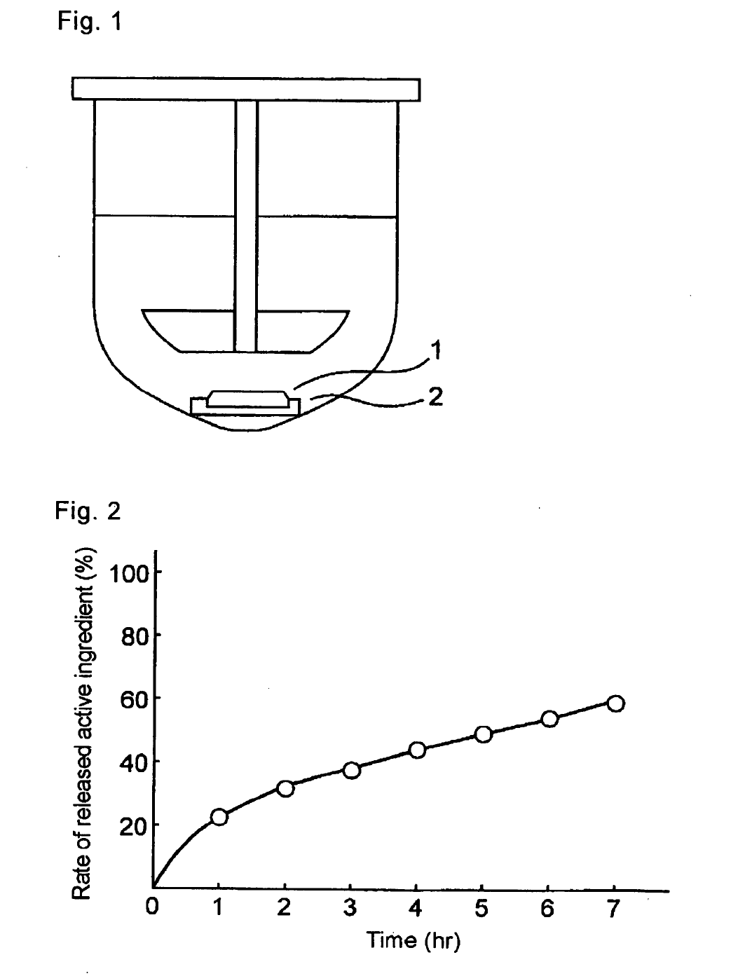

An elution test was performed according to a test method of Japanese Pharmacopoeia, Elution method, Second method with partial modification. That is, about 500 mg of the Composition 1 (1) was filled into a cell having an inner diameter of 30 mm and a depth of 0.5 mm (2) in place of a sinker, and the cell was immersed in the vessel (FIG. 1). And, the liquid was 500 ml of water, and rotation was performed at 100 rpm, 37° C. A concentration of minocycline hydrochloride eluted was calculated by measuring an absorbance at 348 nm with a spectrophotometer (Shimadzu Corporation, UV-260). And, quantification disturbance with a base upon the measurement was not observed. The result thereof is shown in FIG. 2. As apparent from FIG. 2, the Composition 1 produced according to the process of the present invention shows a suitable release behavior and it is demonstrated that the active ingredient may be sustainedly-released as shown by such as 20-40% at 3 hour and 50-60% at 7 hour.

### EXPERIMENTAL EXAMPLE 5

### Microscopic Observation

The composition 1 was directly observed with a phase-contrast microscope, and the Composition 1 diluted with triacetin was observed with a fluorescent microscopy. In addition, the Composition 1 to which 4% aqueous uranyl acetate solution had been added was observed with a scanning electron microscope (Akashi Manufacture Co., Ltd., WET-SEM WS-250, accelerating voltage 25 kV).
When a sustained-releasing pharmaceutical composition (Composition 1) was observed with the phase-contrast microscope and the fluorescent microscopy, there were particles of a diameter of around several micrometers densely, and emission due to minocycline hydrochloride was observed under fluorescent light. Moreover, when 0.001% of Acid Red (a pigment soluble in glycerin which was used as a solvent for hydroxyethyl cellulose for forming the hydro gel) was added to the compositions, the fluorescence was observed at an inside of the particle (not shown in Figure). Therefore, it was found that what forms the particle is a hydro gel composed of glycerin.

### EXPERIMENTAL EXAMPLE 6

### Measurement of Particle Diameter Distribution

It was thought that the hydro gel was dispersed as a particle in the Composition 1 produced in the Production Example 1. Then, a particle diameter distribution of the particle was measured with a centrifugal-type particle diameter measuring apparatus (SA-CP3, SHIMADU Co., Ltd.). A sample for measurement was prepared by diluting the Composition 1 with acetone, shaking the dilution for 1 minute, and dispersing this by sonication for 3 minutes. The sample was measured with the apparatus at an initial speed of 500 rpm and an acceleration of 120 rpm/min. The result thereof is shown in FIG. 3. As apparent from FIG. 3, it was found that the particle diameter of the Composition 1 was 1-10 μm, and an average particle diameter was 4.15 μm. From the foregoing, it was found that the Composition 1 has a structure in which the hydro gels are packed in a closest manner and a triacetin solution containing Eudragit®RS is present in a gap between the hydro gels.

### EXPERIMENTAL EXAMPLE 7

### Mechanism and Control of Sustained-Releasing

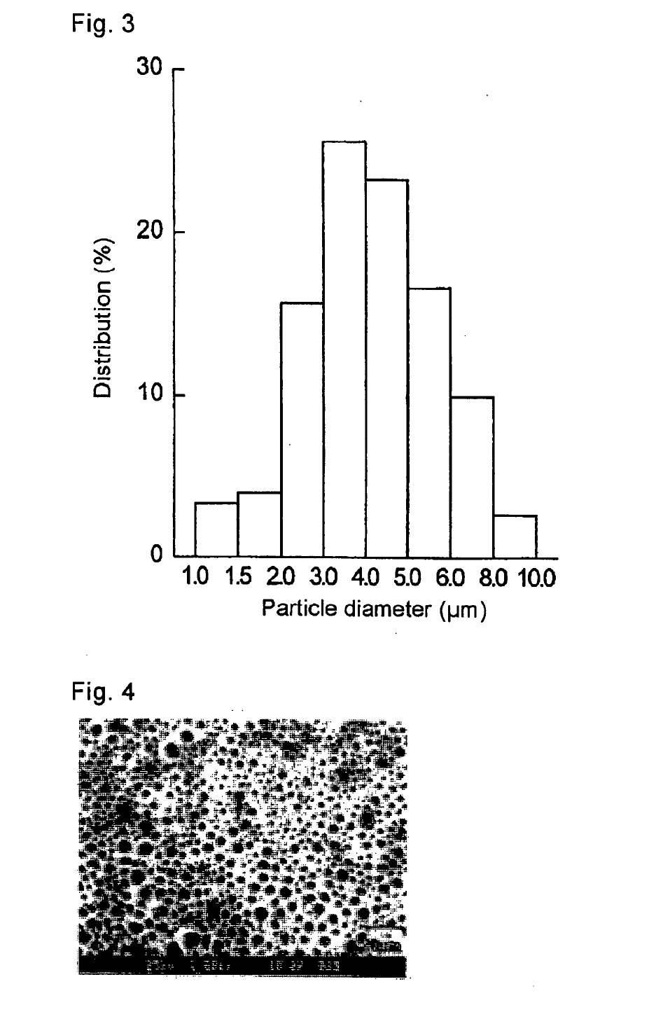

In order to study the state of the Composition 1 upon contact with water, the composition was treated with an aqueous uranyl acetate solution and then observed with a scanning electron microscope. As the result, a network structure as shown in FIG. 4, which was believed to be formed from Eudragit®RS, was observed. Together with observation of other experiments, it is believed that a water-insoluble film of Eudragit®RS (formation of a microcapsule complex) is formed as a mechanism of sustained-releasing exhibited by the Composition 1. Specifically, it is believed that the water-insoluble film of Eudragit®RS is formed by dissolving triacetin in a small amount of water when an Eudragit®RS solution in triacetin, constituting the outside of the hydro gel, is contacted with water and, thereby, the active ingredient contained in the hydro gel is sustainedly-released through the film.

Next, a relationship between a particle diameter and a release rate of the hydro gel was investigated. The particle diameter of the particle was controlled by maintaining a concentration of Eudragit®RS while a concentration of triacetin was increased. As the result, it was found that the particle diameter of the hydro gel became large with increasing the concentration of triacetin.

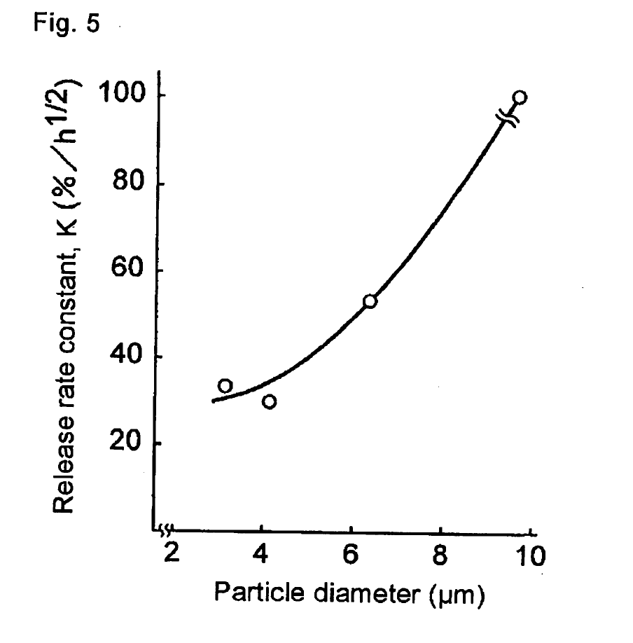

Moreover, as shown in FIG. 5, the release rate constant calculated by a Higuchi's method (T. Higuchi, J. Pharm. Sci., 51, 802 (1962)) also became great with increasing the particle diameter.
Although this phenomenon is contrary to the generally recognized relationship between a particle size of the hydro gel and a dissolution rate, it can be explained as follows. That is, it can be thought that, when the particle diameter of the hydro gel becomes small, an area of the hydro gel contacted with Eudragit®RS per unit volume becomes large, and film formation on the hydro gel with Eudragit®RS is adequately achieved.

### EXPERIMENTAL EXAMPLE 8

### Test of Retention in Gingival Groove

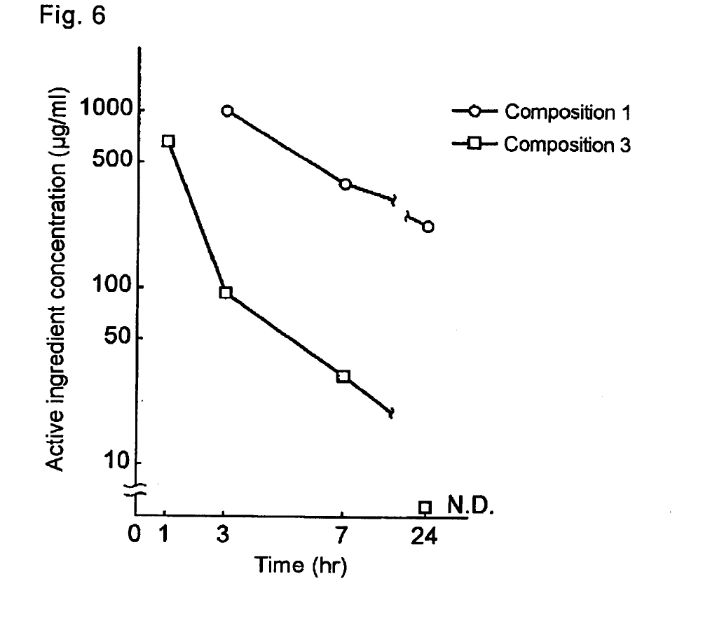

The Compound 1 (microcapsule complex-forming composition) or the Compound 3 (hydro gel-forming composition) was administered to three and four teeth of each of four volunteers having gingival grooves of 1-2 mm depth by using a root canal syringe (Neo Dental Chemical Products Co., Ltd., Tokyo, Japan) until it was overflowed. An administered amount of the Compositions was about 50 mg/tooth. For the volunteers, the normal eating and drinking after administration was permitted, but brushing of teeth was not permitted.
Four paper strips (1.5 mm×12 mm, Advantec MFS, Inc., Tokyo, Japan) were inserted into the gingival groove per one administered tooth and effusion was collected at every constant time after administration. Thereby, a concentration of the remaining active ingredient was measured. The amount of effusion was calculated from a weight difference of the paper strip between before and after insertion.
The collected paper strips were agitated in a mobile phase for 60 minutes, and an amount of the active ingredient extracted from the paper strips was quantitatively measured with HPLC. The condition of HPLC was as follows: pump: Model 510 (Nihon Millipore K.K.), column: Vydac C-18 (Grace Vydac), mobile phase: 16% aqueous isopropyl alcohol solution containing 50 mL of diethanolamine and 1 mM EDTA, adjusted to pH 8 with 1N aqueous sodium hydroxide solution, detector: M490 (Nihon Millipore K.K.), detection wavelength: 345 nm, flow rate: 1.0 ml/sec, injection amount: 50 μl). A result of a measured concentration of the drug after administration into human gingival groove is shown in FIG. 6.
In the case where the Composition 3 was administered, although 640 μg/ml effusion of the active ingredient remained after 1 hour, the concentration was suddenly decreased afterwards and became 32 μg/ml after 7 hours. After 24 hours, the active ingredient could not be detected. On the other hand, in the case where the Composition 1 was administered, it was observed that a high concentration of minocycline hydrochloride such as 387 μg/ml after 7 hours and 228 μg/ml after 24 hours remained. Therefore, it can be said that a smaller time of administration of the Compound 1 than that of the Compound 3 can be expected.

### INDUSTRIAL APPLICABILITY

The present invention belongs to the field of pharmaceutical composition production, and can produce a pharmaceutical composition which can stably contain an active ingredient unstable against water and can sustainedly-release it. Since there are a number of medicinal ingredients, including tetracycline antibiotics, stability of which is deteriorated in the presence of water, a technology for stably formulating such ingredients into the pharmaceutical composition is useful for a pharmaceutical industry. Moreover, sustained-release of such the medicinal ingredient can exhibit efficient potency for a long period of time. Therefore, the present invention is useful for the pharmaceutical industry also in this point.

What is claimed is:

1. A process for producing a pharmaceutical composition, comprising steps of:
mixing and heating a first phase, prepared by mixing following ingredients (A) and (B), and a second phase containing an ingredient (C) under a reduced pressure, before evaporating substantially all water in the first phase by mixing and heating a mixture of first and second phases under a reduced pressure or after evaporating substantially all water in the first phase by mixing and heating the first phase under a reduced pressure; and
adding a third phase containing an ingredient (D) to the mixture to obtain the pharmaceutical composition,
(A) one or more of polyhydric alcohols selected from the group consisting of glycerin, ethylene glycol, diethylene glycol, propylene glycol, dipropylene glycol, hexylene glycol, 1,5-pentanediol, 1,3-butylene glycol, and polyethylene glycol;
(B) one or more of salts selected from the group consisting of magnesium, calcium, and barium salts and hydrous salts thereof;
(C) one or more of water-soluble polymers selected from the group consisting of hydroxyethyl cellulose, hydroxymethyl cellulose, methyl cellulose, hydroxypropyl cellulose, hydroxypropylmethyl cellulose, carboxymethylethyl cellulose, sodium carboxymethy cellulose, sodium alginate, polyvinyl alcohol, polyvinylpyrrolidone, carrageenan, xanthan gum, locust bean gum, guar gum, tragacanth gum, starch and succinoglucan; and
(D) one or more of active ingredients unstable against water.

2. The process according to claim 1, wherein the first and second phases are mixed and heated after evaporating substantially all water in the first phase by mixing and heating the first phase under a reduced pressure.

3. The process according to claim 1, wherein the salt (B) is one or more selected from the group consisting of magnesium chloride, magnesium acetate, magnesium sulfate, magnesium nitrate, magnesium carbonate, magnesium gluconate, magnesium oxide, magnesium hydroxide and hydrous salts thereof.

4. The process according to claim 1, wherein the water-soluble polymer (C) is one or more of cellulose derivatives selected from the group consisting of hydroxyethyl cellulose, hydroxymethyl cellulose, methyl cellulose, hydroxypropyl cellulose, hydroxypropylmethyl cellulose, carboxymethylethyl cellulose and sodium carboxymethy cellulose.

5. The process according to claim 1, wherein the active ingredient (D) is one or more of tetracycline antibiotics selected from the group consisting of tetracycline, minocycline, doxycycline, oxytetracycline, chlortetracycline and pharmaceutically acceptable salts thereof.

6. The process according to claim 1, further comprising a step of adding and mixing a fourth phase, prepared by mixing following ingredients (E) and (F):
(E) methacrylate copolymer; and
(F) one or more of organic solvents selected from the group consisting of triacetin, tributyrin, ethylene glycol diacetate, diethyl sebacate, diethyl phthalate, dibutyl phthalate, diisopropyl adipate, dibutyl succinate, triethyl citrate, N-methyl-2-pyrrolidone and propylene carbonate.

7. The process according to claim 6, comprising steps of:
(1) mixing and heating the first phase under a reduced pressure to evaporate substantially all water therein;
(2) adding the second phase to the first phase and mixing and heating them;
(3) adding the third phase to the mixture and mixing them; and
(4) adding the fourth phase to the mixture and mixing them to obtain a pharmaceutical composition.

8. The process according to claim 6, comprising steps of:
(1) mixing and heating the first phase under a reduced pressure to evaporate substantially all water therein;
(2) adding the second phase to the first phase and mixing and heating them;
(3) adding the fourth phase to the mixture and mixing them; and
(4) adding the third phase to the mixture and mixing them to obtain a pharmaceutical composition.

9. The process according to claim 6, comprising steps of:
(1) mixing and heating the first and second phases under a reduced pressure to evaporate substantially all water therein;
(2) adding the third phase to the mixture and mixing them; and
(3) adding the fourth phase to the mixture and mixing them to obtain a pharmaceutical composition.

10. The process according to claim 6, comprising steps of:
(1) mixing and heating the first and second phases under a reduced pressure to evaporate substantially all water therein;
(2) adding the fourth phase to the mixture and mixing them; and
(3) adding the third phase to the mixture and mixing them to obtain a pharmaceutical composition.

11. The process according to claim 6, wherein the polyhydric alcohol (A) and the organic solvent (F) are immiscible.

12. A pharmaceutical composition produced by a process as defined in claim 1.

13. A pharmaceutical composition produced by a process as defined in claim 6, which produces a microcapsule complex by coming in contact with water.

14. The pharmaceutical composition according to claim 12, which is a dental pharmaceutical composition.

15. The pharmaceutical composition according to claim 14, which is a pharmaceutical composition for treating periodontal diseases.
